(19) United States
(12) Patent Application Publication
(10) Pub. No.: US 2026/0165915 A1
(43) Pub. Date: Jun. 18, 2026

(54) COMPOSITIONS, AGENTS AND PREPARATIONS FOR PROMOTING HAIR GROWTH AND/OR MITIGATING HAIR LOSS

(71) Applicant: LIFE CODE LIMITED, Hong Kong (CN)

(72) Inventor: Wing Hong Lam, Hong Kong (CN)

(21) Appl. No.: 19/536,313

(22) Filed: Feb. 11, 2026

Related U.S. Application Data
(63) Continuation-in-part of application No. PCT/IB2024/057808, filed on Aug. 12, 2024.

(30) Foreign Application Priority Data
Aug. 11, 2023 (HK) 32023077516.7

(51) Int. Cl. A61Q 7/00

(52) U.S. Cl. CPC A61K 8/00; A61Q 7/00

(57) ABSTRACT

Compositions, agents and preparations for promoting hair growth and/or mitigating hair loss. The composition has a backbone structure:

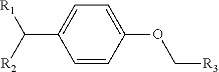

### CROSS-REFERENCE TO RELATED APPLICATIONS

This application is a continuation-in-part application of international patent application No. PCT/IB2024/057808, which was filed on Aug. 12, 2024, published as WO2025037230A1, and claims priority from Hong Kong Short-Term Patent Application No. 32023077516.7, which was filed on Aug. 11, 2023. The contents of all of the aforementioned applications are incorporated by reference herein in their entirety.

### FIELD OF DISCLOSURE

The present disclosure relates to compositions, agents and preparations for human and animal wellbeing, including promoting hair growth and/or mitigating hair loss.

### BACKGROUND

Hair is a feature that is characteristic of mammals. Hair loss, or alopecia, is a concern of many people, as hair is a characteristic of mammals.

### DISCLOSURE

A hair has a hair root and a hair shaft which extends from the hair root and projects upwards to protrude from the skin. The hair root is located inside the skin and surrounded by a hair follicle (“HF”). The hair follicle is a living organ often found in the top two layers of the skin, that is, the dermis and the epidermis. The hair follicle has a sheath of skin and connective tissue, and is connected to a sebaceous gland and a tiny muscle (arrector pili) that helps the hair to stand up.

The hair follicle comprises dermal (mesenchymal) and epidermal (epithelial) cells and hair follicle dermal papilla cells (“HFDPCs”, “HFDP cells”, or “DPC” in short) are a population of mesenchymal cells that reside just under the hair follicle and have the ability to induce new hair follicle formation. Stem cells of the hair follicle are in close proximity to the dermal papilla.

At the base of the hair, the hair root widens to end at a round hair bulb. The hair papilla, which is made of connective tissue and contains nerve endings from the dermis and blood capillaries for supplying blood to the hair root, is also known as derma papilla and found inside the bottom of the hair bulb. Hair starts growing at the bottom of a hair follicle, and new hair cells are constantly being made in the hair bulb, close to the hair papilla.

Hairs grows in cycles, and each cycle has three phases, namely, i) the anagen phase in which the hair begins to grow from the hair root, ii) the catagen phase in which hair growth slows down and the follicle shrinks, and iii) the telogen phase when old hair falls out and new hair begins to grow from the same hair follicle.

Researches show that the anagen phase, which is a growth phase, usually lasts between three and seven years, the catagen phase, which is a transition phase, usually lasts between two and four months, and the telogen phase, which is a resting phase, usually lasts between three and four months; and the telogen phase is believed to be crucial to the formation of healthy hair because a lot of cellular activity happens during this phase so that tissues and regenerate and grow more hair.

Studies show that the hair follicle regulates hair growth via a complex interaction between hormones, neuropeptides, and immune cells, and hair growth begins in the hair root, and have suggested that, during hair growth, signaling molecules including Wnts, BMPs, noggin, and FGFs from the dermal papilla activate hair follicle stem cells (HFSC) to begin proliferating.

Studies show that HFDPCs play an important role in the formation and growth of new hair follicles and hence new hair.

An objective of the present disclosure is to alleviate alopecia, for example, by enhancing growth and/or proliferation of hair, for example by prolonging the hair growth cycles. Prolonging a hair growth cycle may be, for example, by prolonging the anagen phase or by delaying commencement of the telogen phase.

The objective may be achieved by activating HFDPC proliferation and differentiation.

The objective may be achieved by promoting growth and/or proliferation of HFDPC.

The objective may be achieved by promoting or upregulating growth factors such as keratinocyte growth factor (KGF, also known as FGF7), platelet-derived growth factor (PDGF) such as PDGF-AA, and/or basic fibroblast growth factor (bFGF, also known bFGF). The aforesaid growth factors play an important role during hair follicle formation, for example, in the middle stage.

Studies show that KGF is an important endogenous mediator of normal hair follicle growth, development and differentiation, and can induce hair follicle proliferation, as well as the differentiation of progenitor cells and promote epithelialization and wound healing.

Studies show that PDGF is a potent mitogen for cells of mesenchymal origin, contributes to the induction and maintenance of the anagen phase in HFs in vivo, promotes the growth of DPCs, and maintains hair follicle inductive ability in vivo. In general, PDGF-AA increases the expression levels of Sox2, ALP and β-catenin.

Studies show that bFGF (or FGF2) upregulates the expression of PDGFRα of DPCs in vitro.

Studies show that DPC treated with both PDGF-AA and FGF2 show an improved ability to maintain hair inductive activity compared with those treated with FGF2 alone.

In other words, the objective may be achieved by enhancing gene expression of one or more hair promoting growth factors, such as KGF, PDGF-AA, and/or bFGF.

The Wnt pathway plays an important role in the early stage of hair follicle morphogenesis, and dysregulation cany lead to maldevelopment of hair growth and hair loss (alopecia).

The objective may be achieved by activating Wnt pathway(s) in HFDPC, for example, via GSK3beta (glycogen synthase kinase-3 beta), beta-catenin, and/or TCF7L1.

A Wnt pathway may be activated to enhance hair growth, for example, by increasing the protein expression of beta-catenin (for example, via the Wnt/beta-catenin pathway), or by suppression of GSK3beta (for example, via the Wnt/GSK3beta pathway), which will result in downregulated gene expression of GSK3beta.

As beta-catenin binds with LEF1, TCF7/TCF7L1/TCF7L2 to stimulate Wnt driven transcription, it is an objective to upregulated TCF7L1 (aka TCF3) gene expression.

The objective may be achieved by inducing enhanced gene expression of SHBG (sex hormone-binding globulin), of TGIF (transforming growth-interacting factor, aka 5′TG3′ interacting factor).

The objective may be achieved by ameliorating DHT/androgen associated harmful effect on hair follicle growth i.e. androgenetic alopecia.

One or more of the above objectives can be achieved by a substance or substances disclosed herein.

A or each substance disclosed herein may be used as an active ingredient of agents or preparations, for example, for promoting hair growth and/or for mitigating hair loss.

The or each substance disclosed herein may be a composition, for example, in solid state.

The agents or preparation may be aqueous or oil based.

The substance may be configured for topical or other applications.

The objectives of promoting hair growth may be achieved by thickening of hair shaft.

That the compositions, agents and preparations are promising for promoting hair growth would be evident from the further disclosure below.

### SUMMARY

Compositions, agents, and preparations for promoting hair well-being are disclosed.

A substance (“Substance”) that can achieve one or more of the above objectives is a cyclic compound expressible in the skeletal form shown in Table 1 below.

TABLE 1 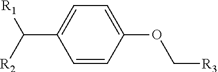
″Substance″,

The Substance is a cyclic compound having a core and a plurality of functional groups R1, R2 and R3 connected to the core.

Specifically, the Substance comprises a hexagonal 6-carbon cyclic structure, a pair of functional groups connected to a first side of the cyclic structure via a carbon bond, the pair of functional groups comprising a first functional group R1 and a second functional group R2, and a third functional group R3 connected to a second side of the cyclic structure that is opposite to the first side via a carbon bond.

The cyclic structure comprises a benzene ring and the functional groups are connected to positions 1 and 4 of the benzene ring via immediate link.

The core has a skeletal structure which is apparently the same as that of 1-Methoxy-4-methylbenzene.

The hexagonal cyclic 6-carbon ring, the oxygen bond connected thereto, and the carbon bond connected thereto may be referred to as a Core herein.

The substance in SMILES expression is: [*]C([*])C1=CC=C(OC[*])C═C1.

R1, R2 and R3 may be selected so that when in combination with the Core, growth or proliferation of human hair follicle dermal papilla cells will be promoted.

R1, R2 and R3 may be selected so that when in combination with the Core, the anagen phase will be prolonged.

R1, R2 and R3 may be selected so that when in combination with the Core, ALP, Versican, beta-catenin, will be enhanced.

At least one of the functional groups R1, R2, R3 is a hydrogen bond donor (“Dn”) or a hydrogen bond acceptor (“Ac”), although two or more of R1, R2, R3 can be hydrogen bond donors, hydrogen bond acceptors, or a combination of a hydrogen bond donor, a hydrogen bond acceptor, hydrogen bond donors and/or hydrogen bond acceptors.

Each one of OH e.g. —COH; —Cn-OH, phenol, —NH2 or —NH1, —CNH(1 to 2) is an example of a hydrogen bond donor.

Each one of ═O, —O—, —OH, —Cl, —Br, —F, —I, —CF (1 to 3), —CN, —N═N, —N≡N, —S, —NO is an example of a hydrogen bond acceptor.

A hydrogen bond donor herein may include an alkyl group or an aryl group.

An alkyl group herein may be an —OH group, such as a —COH; a —Cₙ—OH group, where n is an integer; an —NH1 group, an —NH2 group; or a —C═NH_{(1 or 2)} group.

An aryl group herein may be a phenol group, which is a hydroxyl group bonded to a carbon atom in a benzene ring.

An example form of the substance may be expressed as shown in Table 2 below:

TABLE 2 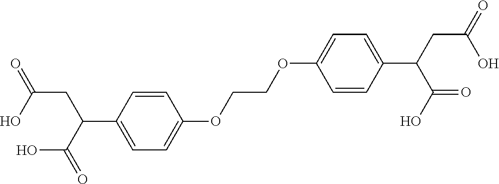 [SMILES: O=C(O)CC(C(=O)O)c1ccc(OCCOc2ccc(C(CC(=O)O)C(=O)O)cc2)cc1]
(LCH 34)

The specific form of the substance of Table 2 is codenamed LCH 34 and has the below characteristics:
2,2′-[Ethane-1,2-diylbis(oxybenzene-4,1-diyl)]dibutanedioic acid
IUPAC name: 2-[4-[2-[4-(1,2-dicarboxyethyl)phenoxy]ethoxy]phenyl]butanedioic acid
InChi Key: HCPDOMFJPYSCGA-UHFFFAOYSA-N
MW: 446.4
SMILES:

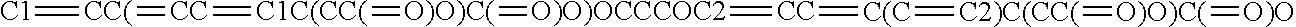

The functional groups R1, R2 and R3 of LCH 34 are as follows:

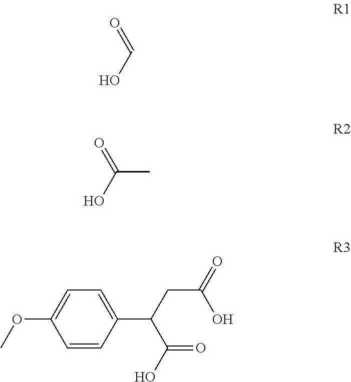

The example substance may be expressed in another specific form as shown in Table 3 below:

TABLE 3 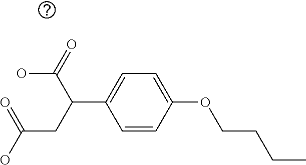 [SMILES: CCCCOc1ccc(C(CC(=O)O)C(=O)O)cc1]
(LCH 67)

The specific form of the substance of Table 3 is codenamed LCH 67 and has the below characteristics:
2-(4-Butoxyphenyl)butanedioic acid
InChIKey: XPMRAXDPYQMFON-UHFFFAOYSA-N
MW: 266.29
SMILES: CCCCOC1=CC=C(C═C1)C(CC(═O)O)C(═O)O

### DESCRIPTION OF FIGURES

The present disclosure is described by way of example and with reference to the accompanying figures, in which:

FIG. 1 is a structural representation of the compositions according to the disclosure.

FIG. 2 is a structural representation of a selected composition which is code-named LCH34.

FIG. 3 shows structural representation of the component groups R1, R2 and R3.

FIG. 4A are data charts showing results of culturing using composition LCH 34 of the present disclosure with reference to ALP protein expression.

FIG. 4B are data charts showing results of culturing using composition LCH 34 of the present disclosure with reference to Versican protein expression.

FIG. 5 shows an image of actual Western blotting result which are used to transcribe into the data of FIGS. 4A and 4B.

FIGS. 6A and 6B are, respectively, images taken on dorsal skin of mice before and after treatment by the composition.

FIG. 6C shows distribution of treatment preparation on dorsal skin of mice of FIGS. 6A and 6B.

FIG. 6D are data charts showing results of treatment on the dorsal skin of mice.

FIG. 7A shows result of hair shaft elongation on ex vivo culturing of human hair follicles using different concentrations of the composition.

FIG. 7B shows distribution of anagen and catagen phases at the end of ex vivo culturing of human hair follicles using different concentrations of the composition.

FIG. 7C shows data on cytotoxicity obtained at the end of ex vivo culturing of human hair follicles using different concentrations of the composition.

FIG. 7D shows data on level of melanin clumping due to the example composition.

FIG. 8A shows data of gene expressions of KGF obtained at the end of culturing demonstrations using a plurality of preparations, each containing the composition.

FIG. 8B shows data of gene expressions of PDGF obtained at the end of culturing demonstrations using a plurality of preparations, each containing the composition.

FIG. 8C shows data of gene expressions of FGF2 obtained at the end of culturing demonstrations using a plurality of preparations, each containing the composition.

FIG. 9 are data charts showing results of culturing using composition LCH 34 of the present disclosure with reference to GSK3beta gene expression.

FIG. 10A are data charts showing results of culturing using composition LCH 34 of the present disclosure with reference to beta-catenin gene expression.

FIG. 10B are data charts showing results of culturing using composition LCH 34 of the present disclosure with reference to beta-catenin protein expression.

FIG. 10C shows an image of actual Western blotting result which are used to transcribe into the data of FIG. 10B.

FIG. 11A shows data of gene expressions of TCF7L1 obtained at the end of culturing demonstrations using a plurality of preparations, each containing the composition.

FIG. 11B shows data of gene expressions of SHBG obtained at the end of culturing demonstrations using a plurality of preparations, each containing the composition.

FIG. 11C shows data of gene expressions of TGIF obtained at the end of culturing demonstrations using a plurality of preparations, each containing the composition.

FIG. 12 shows data of gene expression of hair growth markers including ALP and Versican and hair promoting growth factors including PDGF-AA, KGF and FGF2 using a plurality of preparations, each containing LCH 67 which is a functional variant of the composition LCH 34.

FIG. 13 shows data of gene expression of hair growth markers including ALP and Versican and hair promoting growth factors including PDGF-AA, KGF and FGF2 using a plurality of preparations, each containing LCH 64 which is a functional variant of the composition LCH 34.

FIG. 14 shows data of gene expression of hair growth markers including ALP and Versican and hair promoting growth factors including PDGF-AA, KGF and FGF2 using a plurality of preparations, each containing LCH 65 which is a functional variant of the composition LCH 34.

FIGS. 15A, 15B, 15C, 15D, are sets of figures showing typical back skin conditions of mice at selected days during the period of a typical hair growth cycle.

FIG. 16 are graphs showing quantitative results of FIGS. 15A, 15B, 15C, 15D.

FIGS. 17A, 17B, 17C, 17D, 17E, are sets of figures of histological sections of the dermal layer showing hair follicles of treated and untreated skin at selected days during the period of a typical hair growth cycle.

FIG. 18 are graphs showing lengths of hair follicle of treated and untreated skin at selected days during the period of a typical hair growth cycle.

FIGS. 19, 20, 21 are respectively screen captures of ¹H NMR spectra of LCH 34a, LCH 34b and LCH34.

### DESCRIPTION OF EMBODIMENTS

An example composition which has promising effectiveness in promoting growth and/or proliferation of HFDPC is disclosed herein.

The example composition consists of a backbone structure as shown in FIG. 1, where symbols R1, R2 and R3 represent constituent groups each of which can either be a bond donor (Dn) or bond acceptor (Ac).

The backbone structure (“Backbone”) of the example composition expressed in SMILE notation is:

[*]C([*])C1=CC=C(OC[*])C═C1, where an asterisk (*) in the backbone structure means a constituent group, which may be an unspecified substituent such as a function group containing either a hydrogen bond donor (Dn) or a hydrogen bond acceptor (Ac).

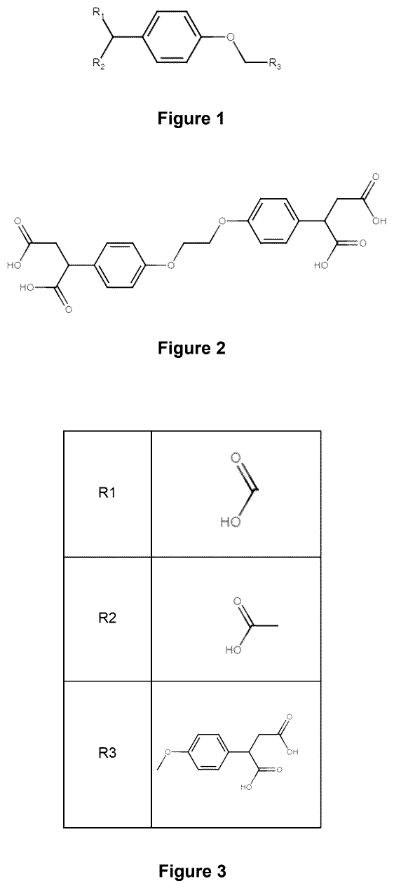

An example composition which has demonstrated effectiveness in promoting growth and/or proliferation of HFDPC and has the backbone structure of FIG. 1 is shown in FIG. 2.

In the example composition of FIG. 2, the constituent groups R1, R2 and R3 have the respective structures shown in FIG. 3. “Structure” herein means chemical structure without loss of generality, unless the context requires otherwise.

The example composition, code-named LCH34, has a molecular weight of 446.4, an IUPAC name of 2,2′-[Ethane-1,2-diylbis(oxybenzene-4,1-diyl)]dibutanedioic acid, an InChi Key is HCPDOMFJPYSCGA-UHFFFAOYSA-N, and a SMILES (Simplified Molecular Input Line Entry System) notation is:

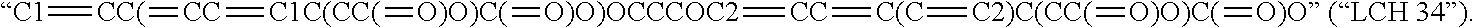

While LCH 34 has the structure of FIG. 2, analyses and/or predictions, for example machine-based analyses and/or predictions, suggested that compositions having the backbone structure are promising in promoting cellular activity of HFDPC such as inductivity and hence hair growth.

That a composition having the backbone structure is promising in promoting hair growth is exemplified through demonstrative culturing of hair follicle dermal papilla cells (HFDPC) using a preparation containing LCH 34. Western blotting performed at the end of the demonstrative culturing shows that culturing of hair dermal papilla cells using LCH 34 resulted in substantial protein expression of ALP and Versican, indicating effectiveness of the composition in promoting hair growth.

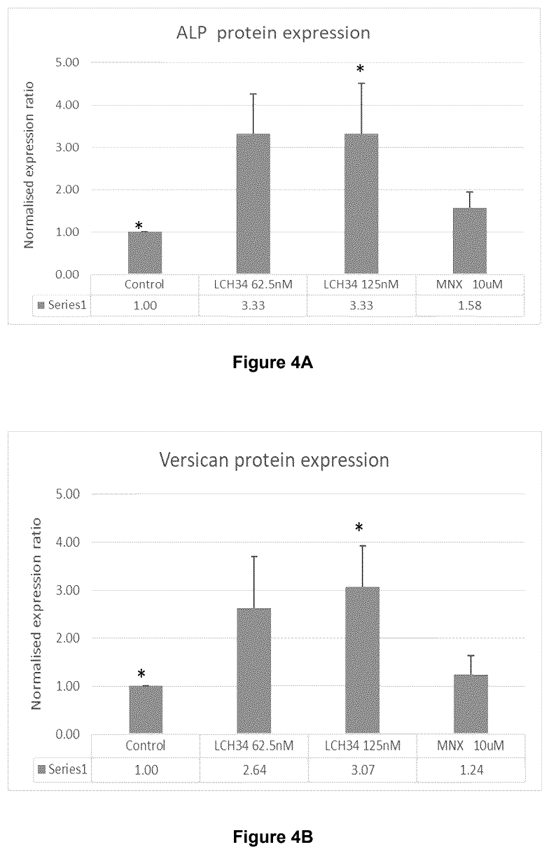

Referring to FIG. 4A, data of the charts show that the protein expressions of ALP resulted from culturing of HFDPC using the composition are significantly higher than the protein expressions of ALP resulted from culturing of HFDPC using a negative control or using a positive control.

Referring to FIG. 4B, data of the charts show that the protein expressions of Versican resulted from culturing of HFDPC using the composition are significantly higher than the protein expressions of ALP resulted from culturing of HFDPC using a negative control or using a positive control.

The demonstrative culturing uses two example concentrations of the composition, namely, a first concentration of 62.5 nM and a second concentration of 125 nM.

The charts of FIG. 4A show that both concentrations give identical results, that is, ALP protein expressions of over 300% compared to the negative control and over 150% compared to the positive control.

The charts of FIG. 4B show that both concentrations of the composition give remarkably good results compared to the negative control and the positive control. More specifically, the composition at the concentration of 62.5 nM resulted in a 264% protein expression and the composition at the concentration of 125 nM resulted in a 307% protein expression compared to the reference protein expression of 100% of the negative control and the 124% protein expression of the positive control.

The demonstrative culturings were performed in an example culturing environment using a vehicle comprising Dulbecco's Modified Eagle Medium (DMEM) as a basal medium, Fetal Bovine Serum (FBS) as supplementation, and carbon dioxide as a pH regulator. The example culturing environment uses 5-10% carbon dioxide to maintain physiological pH of the culturing environment, and an example concentration of 10% FBS. The example culturing environment was provided inside an incubator, with an example culturing period of 3 days.

In the demonstrative culturings, the vehicle is used as a culturing medium for negative control, the vehicle plus minoxidil (MNX) 10 μM is used as a positive control, the vehicle with the composition at a first concentration of 62.5 nM is used as a first demonstration example, and the vehicle with the composition at a second concentration of 125 nM is used as a second demonstration example.

To demonstrate the effectiveness of the composition, immortalized human hair follicle dermal papilla cell lines were cultured using the different types of culturing medium in the culturing environment provided by the incubator for the culturing period.

To evaluate the quantitative proliferation of HFDPC at the end of the culturing period, protein markers such as derma papilla markers are selected and Western blotting is used.

ALP has been known to be associated with hair follicle inductivity in human derma papilla and ALP is regarded as a key indicator of human dermal papilla cells trichogenic (hair forming) inductivity because a maximal level of ALP is always detected in early anagen.

Versican is a chondroitin sulfate proteoglycan which is implicated in the induction of hair morphogenesis, and expressed in the anagen phase of the hair cycle.

Therefore, ALP and Versican were selected as example markers for Western blotting.

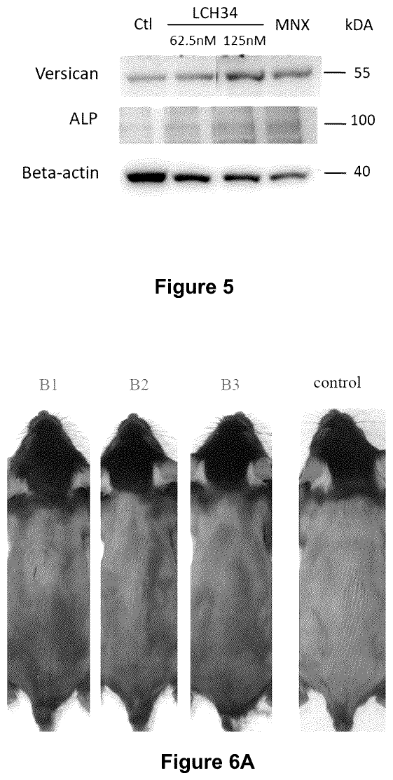

FIG. 5 shows images of Western blotting from which the data of FIGS. 4A and 4B were derived. The Western blotting images have 12 cells which are organised into a matrix of 3 rows and 4 columns. Each row is a protein signal band and the 3 signal bands consist of a first, upper, band on Versican (kDa=55), a second, middle, band on ALP (kDa=100), and a third, lower, band on Beta-actin (kDa=40). The 4 columns consist of a first, leftmost, column on negative control (CTL) of using the vehicle only, a second column on LCH34 of 62.5 nM, a third column on LCH34 of 125 nM, and a fourth, rightmost, column on a positive control of using MNX at 10 μM in the vehicle.

The Western blotting arrangement is configured such that the signal intensity appearing in each cell is proportional to the concentration of the target protein, and the signal intensity is represent in terms of darkness such that a darker signal represents a higher quantitative level of the target protein and vice versa. The beta-actin band is used as a reference to normalize the expression of the target proteins, which are ALP and Versican.

### Demonstrations Using Mice

Mice are a mammal and have a mammalian skin which is widely used as a model system for studying hair growth and hair cycles. That the example composition is promising in promoting hair growth in human beings were also demonstrated using mice as a demonstration model.

While several mouse strains have been used for study of hair growth, the most commonly used type being C57BL/6 and C3H, whose pelage skin pigmentation is known to be merely dependent on their follicular melanocytes. The C57BL/6 mouse was selected as a demonstration example because its dorsal hair has a time-synchronized hair growth cycle. The dorsal skin of a C57BL/6 mouse in the telogen phase is pink and turns dark along with anagen initiation.

To demonstrate the effectiveness of the example composition in the promotion of growth of hair and/or mitigation of hair loss in mammalian skin, the dorsal skin of a plurality of C57BL/6 mouse was depilated to put it in the telogen phase. Some of the mice was used as target samples while the remaining one or ones were used as control. The target samples were applied topically with a daily dose of the example composition on a regular basis during a cultivation period and no example composition was applied to the control sample(s). Hair follicle formation status was measured at the end of the cultivation period to determine the effectiveness of the example composition.

In the demonstrative examples, two example formulations of the example composition, each formed by dissolving the example composition in phosphate buffered saline (PBS) to form an example solution were used. The two example solutions comprise a first example solution having a first concentration of 125 μM and a second example solution having a second concentration which is a higher concentration. The second concentration was 1 mM, that is, eight times the first concentration.

The demonstrations were performed using the example procedures below.

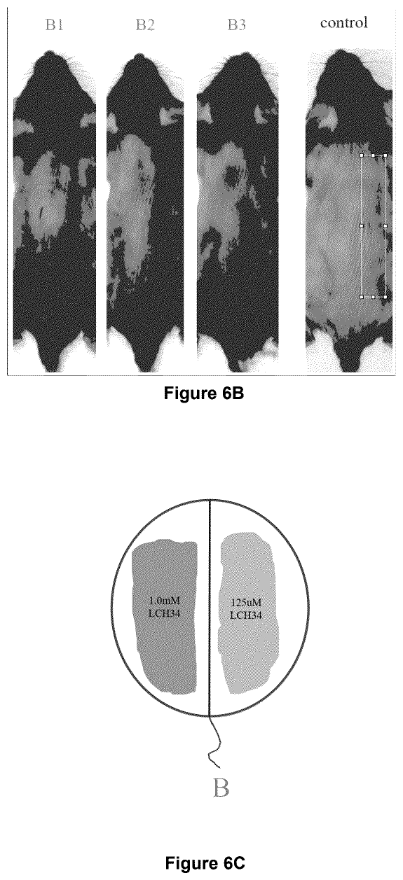

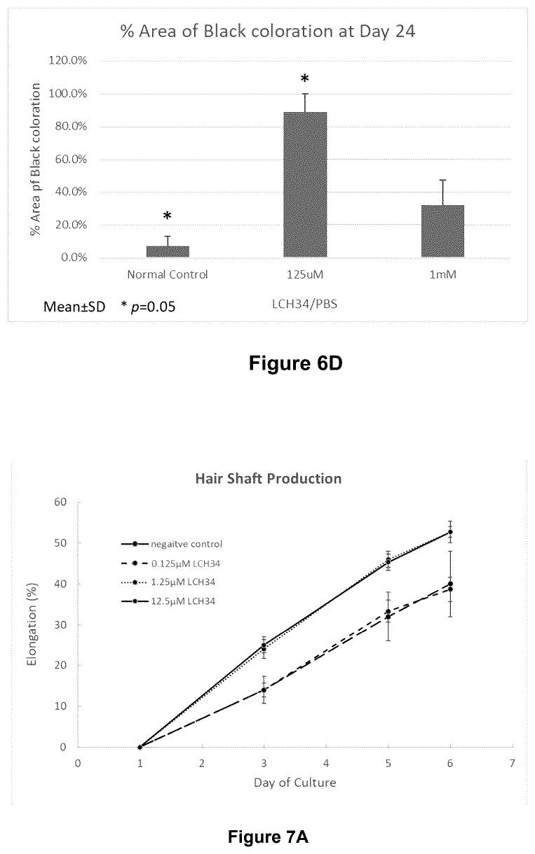

On day 0, which is the beginning of an example observation period, a plurality of black C57BL/6 mice was depilated to leave a bare dorsal skin. Among the mice depilated, some (B1, B2, B3) were used as target samples and one was used as control. A target sample is one to which the example composition was applied, while a control sample is applied with phosphate buffered saline (PBS) which is vehicle of the example solution. Images showing the dorsal skin of the depilated mice B1, B2, B3 and control are excerpted in FIG. 6A.

The example composition was applied to the target samples regularly on a daily basis between day 1 and day 23.

On day 24, which is at the end of the example observation period and when the control sample was expected to enter the telogen phase again, the hair follicle states on the dorsal skin of the plurality of mice was evaluated.

Since studies have shown that the dorsal skin of the mice will turn from white/pink to grey/black on converting from the telogen phase to the anagen phase, whether the dorsal skin or a portion thereof is in the telogen phase or the anagen phase can be determined with reference to the colour, darkness or brightness of the dorsal skin.

To evaluate the hair follicle status, dorsal skin images of the mice were captured and analysed, for example, at the beginning and end of the observation period.

An example way to evaluate hair follicle status of the mice is to measure the brightness or darkness level of the dorsal skin, and to determine whether the dorsal skin is in the telogen phase or the anagen phase according to the measured brightness or darkness levels.

Referring to FIG. 6B, it is observed that a substantial portion of the dorsal skin of the target sample is in the anagen phase, while almost the entire dorsal skin of the target sample is still in the telogen phase. In this regard, a darkness level reaching a threshold is interpreted as corresponding to the anagen phase while a darkness level below the threshold is interpreted as corresponding to the telogen phase.

In the example demonstration, the example solutions were applied such that the first example solution was applied to a first half (right side) of the dorsal skin and the second example solution was applied to a second half (left side) of the dorsal skin, as shown in FIG. 6C.

The results of the demonstrations, expressed quantitatively, are shown in FIG. 6D.

It is noted that while the percentage of anagen regions on the left side of the target samples are substantially higher than that on the control sample, the percentage of anagen regions on the right side of the target samples is substantially higher than that on the left side.

More specifically, the demonstration results show that there is 88.75% coverage of anagen phase on the right side, compared to a 7.14% coverage on the control, indicating an effectiveness of more than 12 times by using the example composition.

As the right side was applied with a lower concentration of 125 μM while the left side was applied with a higher concentration of 1 mM, initial predictions suggest that a concentration of lower than 1 mM is effective in prolonging the anagen phase of hair follicles in the mice.

### Further Studies

In a set of studies, example solutions having different concentrations of the composition were obtained, and mice of the strain C57BL/6 at an age of 8 weeks old were used as subjects of observation. The mice are divided into an example plurality of n=4 groups with each group comprising an example plurality of m=5 mice.

The example solutions were obtained by dissolving the composition in a buffer solution. The example buffer solution used was 10% glycerol/Phosphate buffered saline (PBS), and the example concentrations of the example solutions are 125 μM, 250 μM and 500 μM of LCH34.

The studies began with depilation of the mice, for example, depilation of the dorsal skin, that is, the back skin, followed by daily application of the example solutions of different concentrations to the different mice, and hair growth statuses of the different mice were noted during a period corresponding to a complete hair growth cycle of the subject.

As studies show that this strain of mice has a typical hair growth cycle period of 24 days, which means a typical mouse is expected to enter the telogen phase on day 24, the example solutions were applied daily and topically to treat the depilated back skin consecutively during the typical hair growth cycle immediately after depilation.

The back skin conditions of the each of the subject mice (for example, in terms of darkness or lightness) were then observed and noted for the complete hair growth cycle, and the back skin conditions were recorded on day 0, day 7, day 14, day 18, day 21 and day 24.

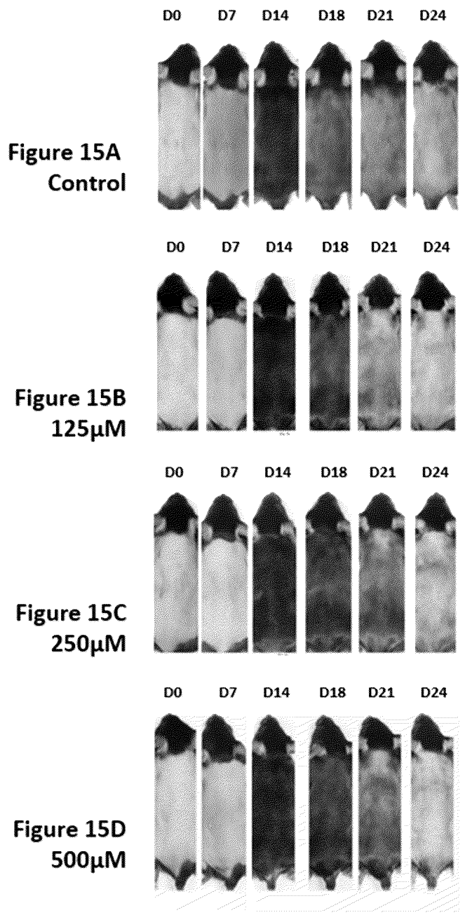

The typical back skin conditions of each group of the subjects during the hair growth cycle are shown in FIGS. 15A, 15B, 15C and 15D, in which FIG. 15A shows those of a group of control subjects with application of the buffer solution but without application of the composition, FIG. 15B shows those of a group of subjects treated with a first concentration in the buffer solution, FIG. 15C shows those of a group of subjects treated with a second concentration in the buffer solution, with the second concentration higher than the first concentration, and FIG. 15D shows those of a group of subjects treated with a third concentration in the buffer solution, with the third concentration higher than the second concentration. In this example, the second concentration is double the first concentration, while the third concentration is double the second concentration.

Referring again to the FIGS. 15A, 15B, 15C and 15D, all the subjects under observation had their dorsal skin fully darkened by day 14, representing that all the subjects are in the anagen phase by then (that is, after the half growth cycle period), and all the darkened skin became lighter by day 21, representing that all the subjects are transitioning from the anagen phase into the catagen phase by then.

However, it is noted that the colour of the dorsal skins of the control is significantly lighter than any of the subjects treated with the composition by day 18, with the subjects treated with the composition still have substantially darkened skin by day 18, signifying a prolongation of the anagen phase by at least 4 days, that is, ⅙ of the hair growth cycle period of 24 days.

This period of prolongation when transcribed into the typical human hair growth cycle period of 6-7 years would correspond to a prolongation of the anagen phase for at least one year.

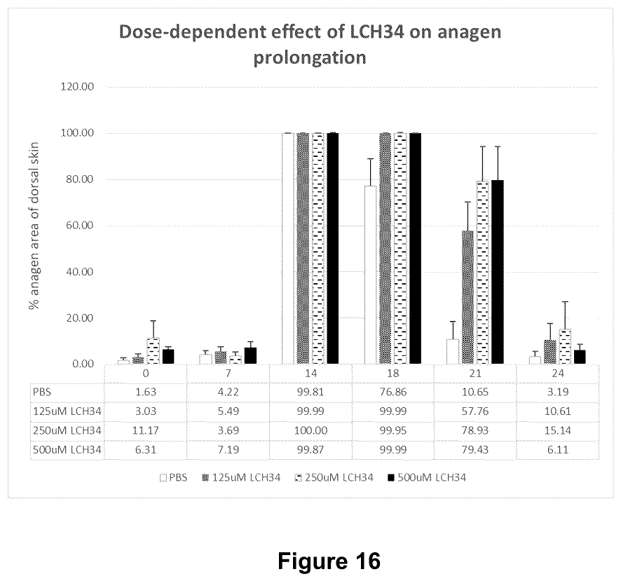

The results of the studies when processed quantitatively are shown in FIG. 16. Some salient features that can be extracted from the quantitative results are as follows:

On day 18, the anagen areas of all the three composition-treated mice are greater than the control by an average of about 30%

On day 21, the mice treated with the second and third concentrations of 250 μM and 500 μM have an average anagen area of 7.4 times to that of the control, while the mice treated with the first concentration of 125 μM has an average anagen area of 5.4 times to that of the control, suggesting that a concentration of between 125 μM and 250 μM is probably sufficiently effective for topical applications.

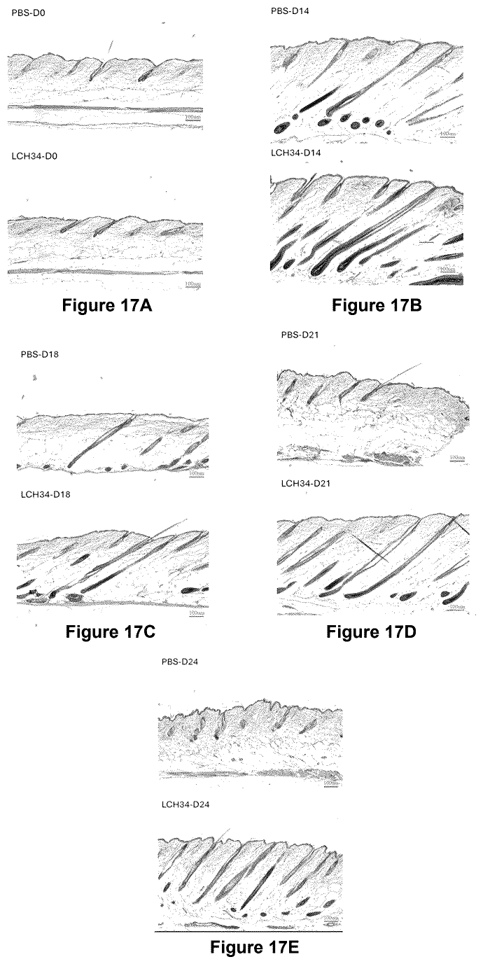

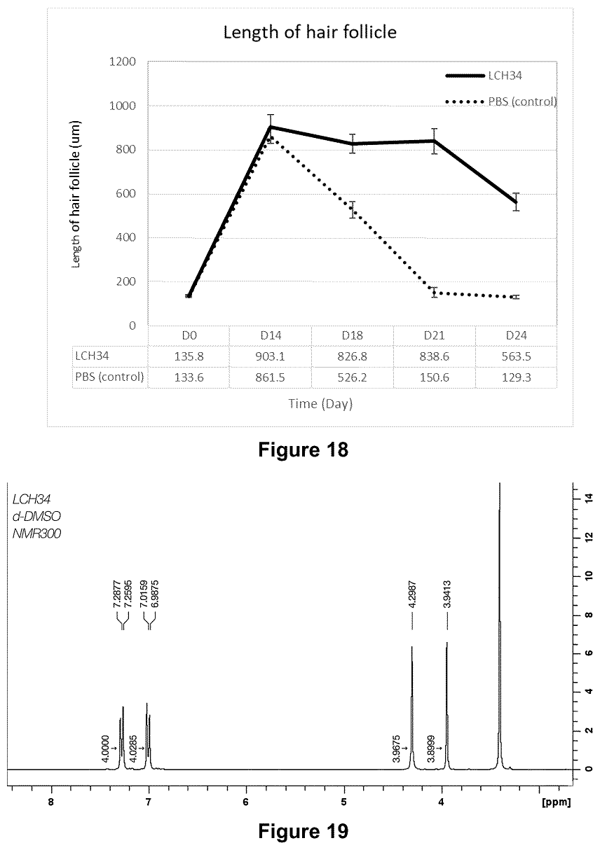

The darkness/lightness results are consistent with the example histological results shown in FIGS. 17A, 17B, 17C, 17D and 17E. For example, it can be deduced from the results of day 18 that the hair follicles of composition treated subject is very much healthier than that of the control, as the hair follicles of the control are substantially shortened and degraded compared to the composition treated subject, as shown in FIG. 18.

Furthermore, it can also be deduced from the results of day 24 that the hair follicles of composition treated subject is still healthy, in that the hair follicles still maintain a good length, and ready to enter the next anagen phase, while the hair follicles of the control are not so ready.

Therefore, it can be deduced from the histological results that the composition treated hair follicles suffer significantly less degradation compared to their untreated counterpart, and that the composition treatment does effectively and significantly extend the duration of the anagen phase or delay entry into the telogen phase.

Studies have shown that dorsal skin of the C57BL/6 mice strain is a good model for studying human hair growth, and the same or comparable concentrations of the composition would be appropriate for human applications.

Effectiveness of the example composition in promoting hair growth for human beings was demonstrated using human hair follicles in ex vivo culture. In example demonstrations using human hair follicles, the human hair follicles (HF) were obtained from informed donors with written consent, and with approval of ethics committee under the Monasterium Laboratory Biobank approval 2019-297-f-S, study plan 2020-954-f-S, and the studies were conducted according to the Declaration of Helsinki principles.

Effectiveness of the example composition in promoting hair growth was demonstrated with ex vivo culturing of human hair follicles using the example composition. The ex vivo culturing demonstrated promising results.

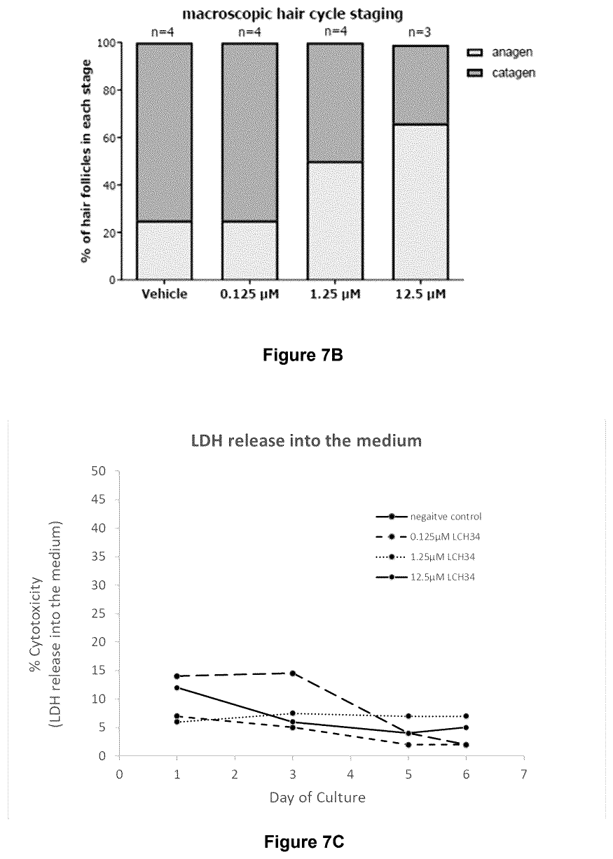

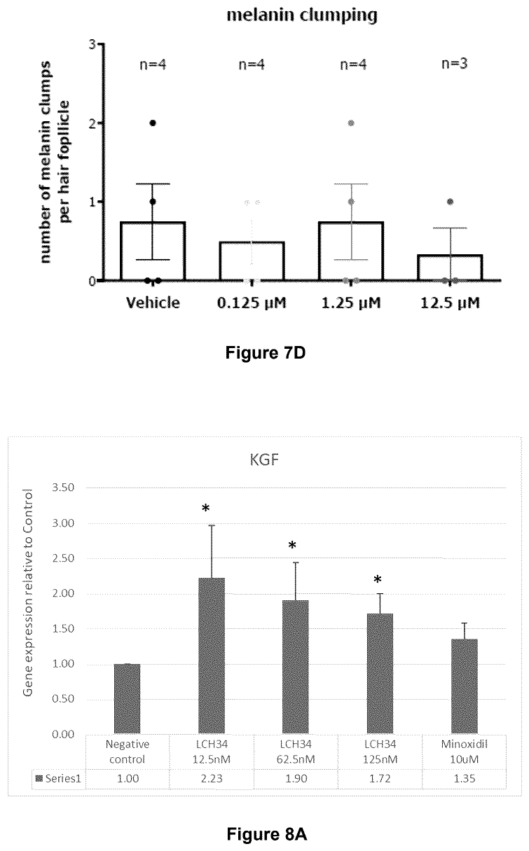

Referring to FIGS. 7A and 7B, the ex vivo cultured hair follicles using the example composition show substantial growth during a culturing period, compared to those of control samples. In the culturing examples of FIGS. 7A and 7B, an example plurality of three example concentrations of the example composition were used to culture the human hair follicles. The three example concentrations of the example composition are a first concentration of 0.125 μM, a second concentration of 1.25 μM and a third concentration of 12.5 μM. The negative control used a culturing medium without adding the sample composition or other known hair growth promotion agents to culture the human hair follicles. A culturing medium without adding the sample composition or other known hair growth promotion agents is also known as “vehicle” to persons skilled in the art.

The results of FIG. 7A show that example hair follicles cultured using the second and third concentrations resulted in a more substantially growth of hair shaft (in terms of increase in length or elongation of hair shaft) compared to their control counterparts, which are control samples labelled “negative control” in FIG. 7A. For example, the hair follicles cultured using a concentration of 1.25 μM has a growth in length of over 20% in 2 days (day 3), over 40% in 4 days (day 5) and about 50% in 5 days (day 6), while the growth in length of the controls samples is slightly more than 10% in day 3, about 30% in day 5 and about 35% in day 6. The hair follicles cultured using a concentration of 12.5 μM has a growth result that is same as, if not identical to, that of 1.25 μM. The hair follicles cultured using a concentration of 0.125 μM has a growth result that is same as, if not identical to, that of the control samples. It can be deduced from the results of FIG. 7A that a concentration of the example composition at more than 0.125 μM, for example, from 1.25 μM to 12.5 μM, is beneficial for promotion of hair growth.

The results of FIG. 7B show that of the samples treated with a concentration of 1.25 μM to 12.5 μM of the example composition, there are substantially more hair follicles in the anagen phase at the end of the culturing period compared to their control counterparts. This suggests that the example composition was effective in prolonging the period of the anagen phase in human hair follicles.

Referring to FIG. 7B, 25% of the hair follicles cultured using a concentration of 0.125 μM of the example composition, 50% of the hair follicles cultured using a concentration of 1.25 μM of the example composition, and 67% of the hair follicles cultured using a concentration of 12.5 μM of the example composition remained in the anagen phase at the end of the culturing period, while only 25% of the control samples was in the anagen phase.

It is deduced from the results of FIG. 7B that a concentration of the example composition at more than 0.125 μM, for example, from 1.25 μM to 12.5 μM, is beneficial for promotion of hair growth, as the duration of the anagen phase is prolonged.

In addition to enhancement of hair growth, the ex vivo culturing also showed that the example composition does not have or does not induce toxicity which needs concern. As evident from FIG. 7C, the cytotoxicity measured with reference to amount of Lactate dehydrogenase (LDH) released into the culturing medium is comparable to that released by the control samples cultured without the example composition.

Referring to FIG. 7C, the percentage of cytotoxicity due to the example composition remains more or less at a constant level of below 10%, while the cytotoxicity of the control counterparts is at or above 10% initially and then drops to below 10% at the end of the culturing period.

The ex vivo culturing also showed that the example composition does not raise concern of melanin clumping. Referring to FIG. 7D, the levels of melanin clumping due to the example composition are comparable or lower than that of the control samples. As a higher level of melanin clumping means a higher level of toxicity, the comparable or lower melanin clumping levels due to the example composition compared to that of the control samples suggest that the example composition does not increase melanin clumping.

### Culturing of Human Hair Follicles Ex Vivo to Evaluate Effectiveness

Effectiveness of the example composition in promoting growth of human hair follicles may be and was demonstrated through ex vivo culturing of human hair follicles using example methodology, materials and environment described herein.

### Culturing Medium

Williams Complete Media (WCM) may be and was used as an example culturing medium for the example ex vivo culturing of HF cells. The culturing medium may be and was formed by supplementing William's E media with 2 mM of L-glutamine (Gibco), 10 ng/ml hydrocortisone (Sigma Aldrich), 10 μg/ml insulin (Sigma Aldrich) and 1% of penicillin/streptomycin mix (Gibco). William's E Medium contains zinc, iron, manganese, non-essential amino acids, the reducing agent glutathione and the lipid methyl linoleate.

### Culturing Environment

The culturing environment may be and was set at 37° C. with 5% CO₂ in an incubator. A 48-well plate may be and was provided in the incubator and each hair follicle sample may be and was allocated an individual well so that each hair follicle is cultured in its own well using minimal culturing medium.

### Culturing Period

A culturing period of 6 days (days 0 to 5) may be and was selected according to past hair follicle culturing experiences. The culturing medium was applied on day 1 and changed on days 3 and 5.

### Hair Follicle Samples

Human scalp hair follicles (HHFs) in anagen stage, for example, anagen stage VI, may be and was selected for ex vivo culturing in example demonstrations.

### Determination of Growth

Studies have shown that growth of human hair follicles and hair shaft production are positively correlated. The growth of an HHF may be determined with reference to elongation. Therefore, the length or elongation of sample HHFs may be and was measured at different times during the culturing period to determine the extent of growth or otherwise of the sample HHFs. The growth of an HHF in ex vivo culturing demonstrations may be and was determined with reference to a base length which is a reference length. The base length may be and was taken as the length of the HHF on day 0. The lengths of the cultured HHFs were then measured on selected intervals to determine growth with respect to the reference. The length of an HHF may be and was determined by measuring between the end of the bulb connective tissue sheath and the end of the distal outer root sheath.

Determination of Hair Cycle Stage with Reference to Macroscopic Parameters

Studies show that prolonging the anagen phase of HHFs has the effect of promoting hair growth, since a direct effect of prolonging the anagen phase is the slowing down of hair falls from the human scalp. Practical experiences have shown that most human hair follicles in the culturing environment would be in the telogen phase at the end of the culturing period. Determining the number or percentage of hair follicles cultured in the culturing environment which is in the anagen phase and/or which is in the telogen phase at the end of the culturing period would provide useful data for evaluating the effectiveness of the example composition in promoting hair growth, for example, terminal hair growth, in human beings.

The hair cycle stage of each hair follicle may be and was determined with reference to one or more macroscopic parameters, for example, by using high magnification (50× and 200×) bright field images. Example macroscopic parameters are set out in Table 4 herein.

TABLE 4
(source: Human hair follicle organ culture: theory, application
and perspectives, Langan et al., Exp. Dermatol 2015)

| Hair cycle stage | Anagen | Catagen |
|---|---|---|
| Hair shaft | Substantial hair shaft | Reduced or no hair |
| production | production at all time | shaft production during |
| | points | the last days of the |
| | | culture |
| Hair bulb shape | Rounded | Bullet-like, slightly |
| | | triangulated, thinned |
| | | and elongated |
| Hair matrix shape | Thick, closed tips | Thin, shortened, |
| and thickness | | opening tips |
| Dermal papilla (DP) | Almond-shaped | More rounded, densely |
| shape | | packed |
| Pigmented hair shaft | Very pigmented, | Less pigmented, |
| | starting at the level of | starting at the level of |
| | Auber's line and expand | Auber's line or above |
| | to half of hair matrix | dermal papilla |

### Determination of Toxicity

Potential toxicity of the example composition on application to human beings may be and was determined with reference to release of LDH or melanin clumping.

Release of LDH into the Culturing Medium

Measurements of lactate dehydrogenase (LDH) in the culturing medium, which would indicate activity due to cytoplasm of damaged HF cells, would provide a good indication of potential cytotoxicity of the example composition. Colorimetric assays (Cytotoxicity Detection Kit (LDH), Roche) may be and was used to quantify cell lysis and the cytotoxic effect.

### Macroscopic Evaluation of Melanin Clumping

Melanin clumping, for example, ectopic melanin clumping, is a sign of hair follicle cytotoxicity and dystrophy (Bodo et al., Am J Pathol 2007). Ectopic melanin clumping may be and was evaluated using merge z-stack images taken with a high-resolution microscope at 20× magnification at the end of the culture period in the hair bulb area within 500 μM from the tip along the shaft.

### Ex Vivo Culturing of Example Human Hair Follicles Using the Example Composition

### Human Hair Follicle Samples

Four groups of human hair follicles (HHFs) were used, and each group comprises 3 or 4 HHFs. 15 HHFs were taken from a 38-year-old hair-healthy Caucasian male donor due to geographical convenience.

### Procedures

On day 0, the HHFs taken from the donor were placed in individual wells in the incubator, which is maintained at the culturing environment. The HHFs were divided into an example plurality of 4 groups, of which group 1 consists of a sample size of 4 HHFs, group 2 consists of a sample size of 4 HHFs, group 3 consists of a sample size of 4 HHFs, and group 4 consists of a sample size of 3 HHFs.

On day 1, some of the groups were treated with the example composition, while one is allocated as a control group and not so treated. Specifically, group 1 consisting of 4 HHFs was selected as a control group, group 2 consisting of 4 HHFs was treated with a first concentration of 0.125 μM, group 3 consisting of 4 HHFs was treated with a second concentration of 1.25 μM, and group 3 consisting of 3 HHFs was treated with a third concentration of 12.5 μM.

On days 3 and 5, old culturing medium was replaced by new culturing medium of same constituents.

On days 0, 1, 3, 5 and 6, various measurements were taken to obtain data and the demonstrations concluded on day 6.

### Mechanistic Studies

### Pertinent Growth Factors

Growth factors are known to be crucial to the regulation of hair cycles and hair growth. Growth factors are polypeptides that regulate growth and differentiation of many cell types. Growth factor families including the epidermal growth factor (EGF)-related ligands, fibroblast growth factors (FGF), transforming growth factor-beta (TGF-beta), insulin-like growth factor (IGF), hepatocyte growth factor/scatter factor (HGF/SF), and platelet-derived growth factor (PDGF) have been shown to be crucial for the regulation of the hair cycle and hair growth and are pertinent growth factors.

Of the various growth factor families, studies have revealed that the FGF family seems to have a more pertinent influence on the growth and development of hair follicles, and Keratinocyte growth factor (KGF or FGF-7), which is a 28-kd member of the FGF family, is known to induce proliferation of a wide variety of epithelial cells, including keratinocytes within the epidermis and dermal adnexa. Because KGF is known to induce proliferation of keratinocytes, KGF is selected as a marker for quantitative evaluation of the effectiveness of the composition.

That the composition is promising in promoting hair growth is demonstrated through culturing of HFDP cells using a culturing medium containing the composition and quantitative evaluation of resulting expressions of various pertinent growth factors.

### KGF

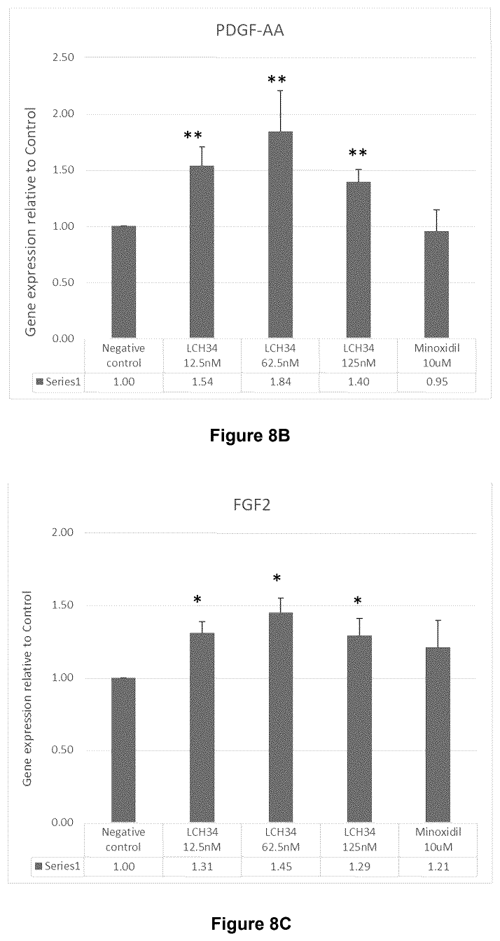

Example culturing of HFDP cells using selected concentrations of the composition in the culturing environment showed substantial gain in gene expression of KGF, as evident from FIG. 8A.

Referring to FIG. 8A, the data show expressions of 223%, 190%, and 172% resulting from culturing of HFDP cells in the culturing environment using culturing preparations containing the composition at concentrations of 12.5 nM, 62.5 nM, and 125 nM, respectively. The results are compared to an expression of 135% resulting from culturing using MNX at 10 μM and an expression of 100% resulting from culturing using the vehicle only, which is set as a reference and a negative control.

The data of FIG. 8A suggest that the expression of KGF may have peaked when the composition is at a concentration of 12.5 nM or below, suggesting a concentration of below 125 nM, below 62.5 nM, below 12.5 nM, etc, would be sufficiently promising.

### PDGF

PDGF is a potent mitogen for cells of mesenchymal origin, and is known to contribute to the induction and maintenance of the anagen phase in HFs in vivo. PDGF was considered to be essential factor to promote the growth of DPCs and to maintain hair follicle inductive ability in vivo. PDGF-AA is a member of the PDGF family which is known to have increase expression levels of Sox2, ALP and β-catenin, all of which are markers of hair growth.

Example culturing of HFDP cells using selected concentrations of the composition in the culturing environment showed substantial gain in gene expression of PDGF-AA, as evident from FIG. 8B.

Referring to FIG. 8B, the data show expressions of 154%, 184%, and 140% resulting from culturing of HFDP cells in the culturing environment using culturing preparations containing the composition at concentrations of 12.5 nM, 62.5 nM, and 125 nM, respectively. The results are compared to an expression of 95% resulting from culturing using MNX at 10 μM and an expression of 100% resulting from culturing using the vehicle only, which is set as a reference and a negative control.

The data of FIG. 8B suggest that the expression of PDGF-AA may have peaked at a concentration between 12.5 nM and 125 nM, and may have peaked in the vicinity of 62.5 nM, for example, ±5 nM, 10 nM, 15 nM, 20 nM, 25 nM, 30 nM of 62.5 nM.

FGF2 (aka bFGF)

FGF2 (aka bFGF) are known to upregulate the expressions of PDGFRα of HFDPCs in vitro. PDGFRα is a kind of PDGF receptors to which PDGF-AA will bind, whereby downstream signal proteins will be triggered, and different types of gene expressions induced, as a result of which proliferation of DPCs are finally promoted.

Example culturing of HFDP cells using selected concentrations of the composition in the culturing environment showed noticeable gain in gene expression of FGF2, as evident from FIG. 8C.

Referring to FIG. 8C, the data show expressions of 131%, 145%, and 129% resulting from culturing of HFDP cells in the culturing environment using culturing preparations containing the composition at concentrations of 12.5 nM, 62.5 nM, and 125 nM, respectively. The results are compared to an expression of 121% resulting from culturing using MNX at 10 μM and an expression of 100% resulting from culturing using the vehicle only, which is set as a reference and a negative control.

The data of FIG. 8C suggest that the expression of FGF2 may have peaked at a concentration between 12.5 nM and 125 nM, and may have peaked in the vicinity of 62.5 nM, for example, ±5 nM, 10 nM, 15 nM, 20 nM, 25 nM, 30 nM of 62.5 nM.

The results of FIGS. 8B and 8C show that the composition induces more gene expressions of PDGF-AA and FGF2. As studies had shown that combining PDGF-AA and FGF2 would have a synergistic effect in promoting hair inductive activity in DPCs, and using the composition to treat DPCs has been shown to enhance expressions of both PDGF-AA and FGF2, suggesting that the composition has a positive synergistic effect in promoting hair growth, (for example, see Kiso M et al., Synergistic effect of PDGF and FGF2 for cell proliferation and hair inductive activity in murine vibrissal dermal papilla in vitro. J Dermatol Sci. 2015 August; 79(2):110-8.).

### Wnt Pathway & GSK3beta

Wnt/beta-catenin pathway is known as one of key pathways to activate HFDPC proliferation and differentiation. When this pathway is turned on, beta-catenin is protected from degradation by suppressing beta-catenin destruction complexes which comprises glycogen synthase kinases 3 beta (GSK3 beta), axin, adenomatosis polyposis coli (APC), and casein kinase 1 alpha (CK1alpha) so that beta-catenin can translocate from cytosol to nucleus to stimulate Wnt driven transcription.

Therefore, enhancement of beta-catenin and/or suppression of GSK3 beta are indicia of effectiveness in facilitating promotion of hair growth.

Data obtained at the end of the ex-vivo culturing showed that the gene expressions of GSK3beta are substantially downregulated.

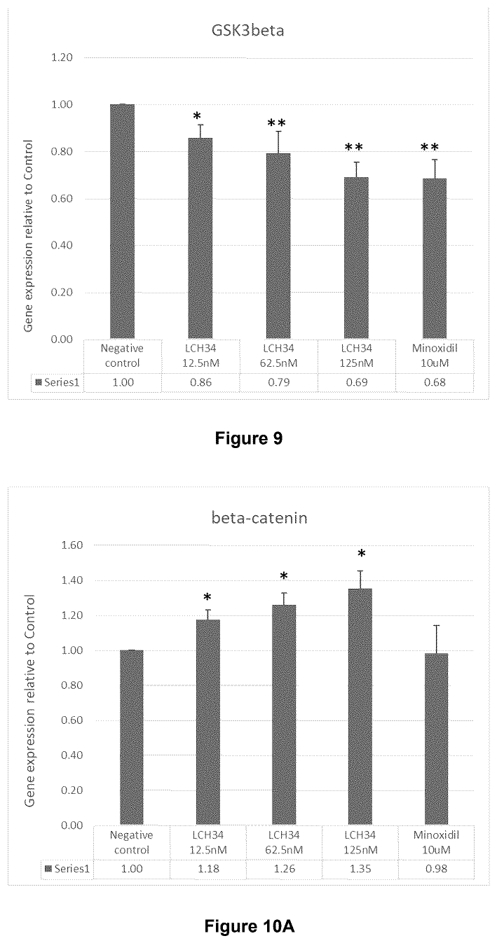

Referring to FIG. 9, the data show that the ex-vivo culturing using the composition at concentrations of 12.5 nM, 62.5 nM, and 125 nM, resulted in downregulation of the gene expressions of GSK3beta to 86%, 79% and 69% respectively, compared to the downregulation to 68% when MNX 10 μM is used and the no downregulation of 100% when the vehicle alone as a negative control is used.

The data of FIG. 9 show a trend which suggests that the downregulation may continue to increase with an increase in concentration of the composition to above 125 nM, although a concentration as low as 12.5 nM already shows a positive downregulation.

Data obtained at the end of the ex-vivo culturing showed that beta-catenin expressions are substantially enhanced.

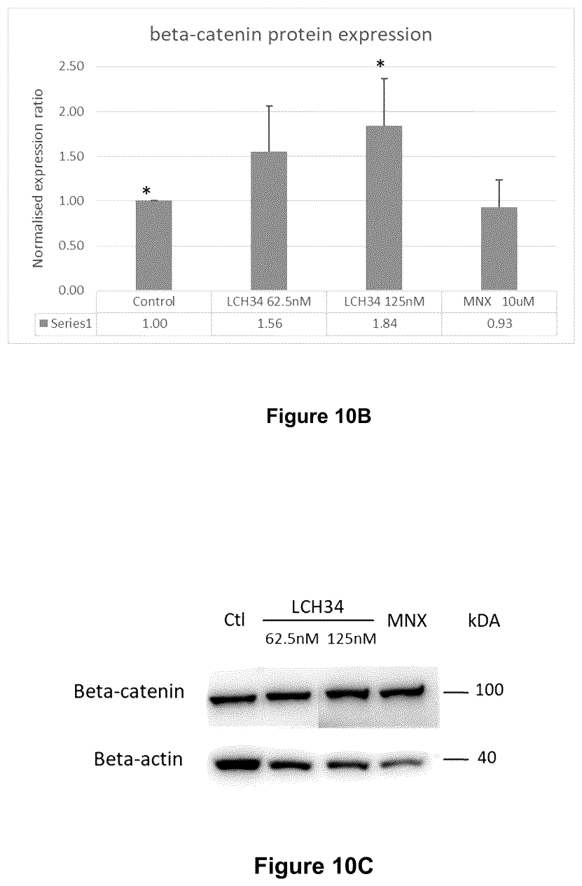

Referring to FIG. 10A, the data show that the ex-vivo culturing using the composition at concentrations of 12.5 nM, 62.5 nM, and 125 nM, resulted in enhancement of gene expressions of beta-catenin to 118%, 126% and 135% respectively, compared to the downregulation to 98% using MNX 10 μM and the no enhancement of 100% using the vehicle alone as a negative control.

The data of FIG. 10A show a trend which suggests that the enhancement may continue to increase with an increase in concentration of the composition to above 125 nM, although a concentration as low as 12.5 nM already shows a positive enhancement effect, which is a better effect than that due to the positive control using MNX.

Referring to FIG. 10B, the data show that the ex-vivo culturing using the composition at concentrations of 62.5 nM and 125 nM, resulted in enhancement of protein expressions of beta-catenin to 156% and 184% respectively, compared to the downregulation to 93% when MNX 10 μM is used and the no enhancement of 100% when the vehicle alone as a negative control is used.

The data of FIG. 10B show that a concentration as low as 62.5 nM already shows a positive enhancement effect which is better than that due to the positive control using MNX, and the positive enhancement effect is even more notable when the concentration is increased above 125 nM.

FIG. 10C shows images of Western blotting from which the data of FIG. 10B were derived. The Western blotting images have 8 cells which are organised into a matrix of 2 rows and 4 columns. Each row is a protein signal band and the 2 signal bands consist of a first, upper, band on beta-catenin and a second, lower, band on Beta-actin (kDa=40) as a control reference. The 4 columns consist of a first, leftmost, column on negative control (CTL) of using the vehicle only, a second column on LCH34 of 62.5 nM, a third column on LCH34 of 125 nM, and a fourth, rightmost, column on a positive control of using MNX at 10 μM in the vehicle.

### Other Relevant Compounds

### TCF7L1:

TCF7L1 is a transcription factor which is known to collaborate with β-Catenin in its established role in hair follicle stem cell activation.

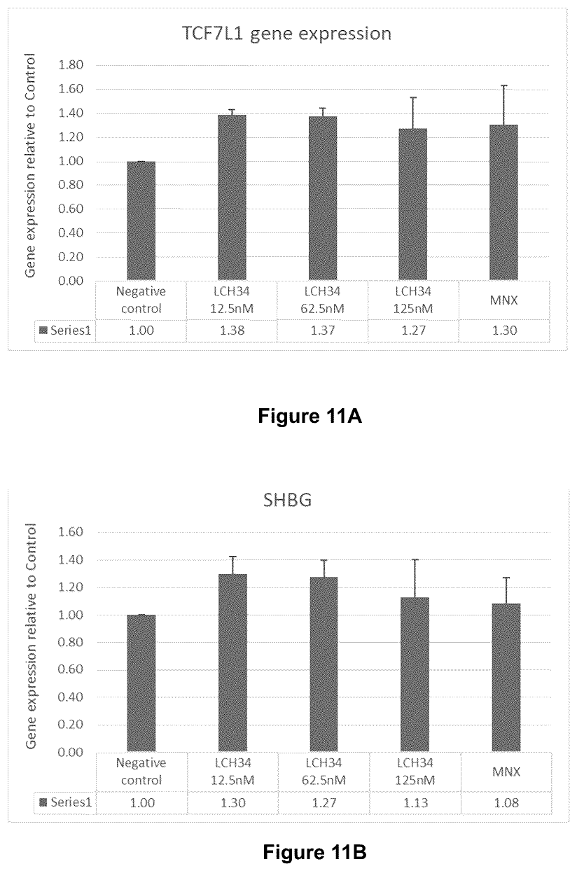

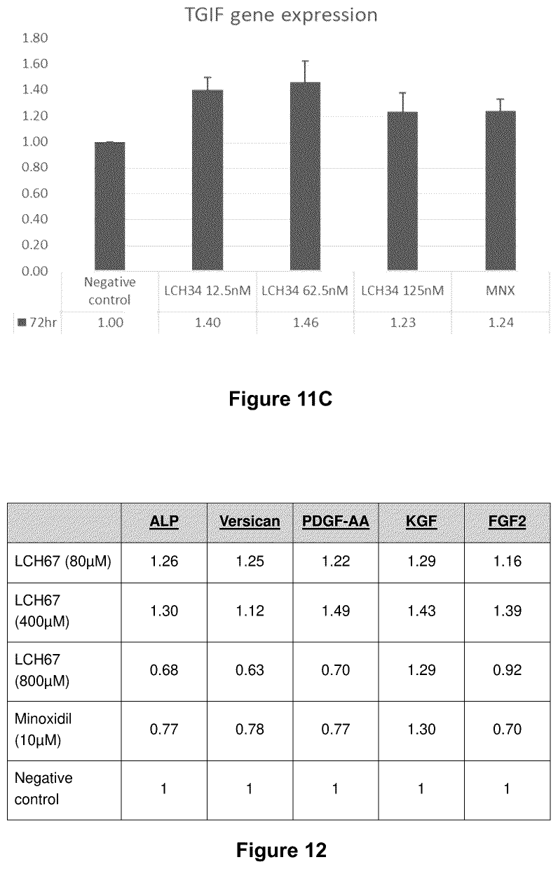

Example culturing of HFDP cells using selected concentrations of the composition in culturing environment showed substantial gain in gene expression of TCF7L1, as evident form FIG. 11A.

Referring to FIG. 11A, the data show expressions of 138%, 137%, and 127% resulting from culturing of HFDP cells in the culturing environment using culturing preparations containing the composition at concentrations of 12.5 nM, 62.5 nM, and 125 nM, respectively. The results are compared to an expression of 130% resulting from culturing using MNX at 10 μM and an expression of 100% resulting from culturing using the vehicle only, which is set as a reference and a negative control.

The data of FIG. 11A suggest that the expression of TCF7L1 may have peaked when the composition is at a concentration of 12.5 nM, suggesting a concentration of below 125 nM, below 62.5 nM, below 12.5 nM, etc, would be sufficiently promising.

### Sex Hormone-Binding Globulin (SHBG):

Sex Hormone-Binding Globulin (SHBG) is a protein which binds to dihydrotestosterone (DHT) and testosterone to limit their bioavailability. Since high level of DHT can damage hair follicles while DHT is produced from testosterone, limiting the bioavailability of them can reduce risk of hair loss.

Example culturing of HFDP cells using selected concentrations of the composition in culturing environment showed substantial gain in gene expression of SHBG, as evident form FIG. 11B.

Referring to FIG. 11B, the data show expressions of 130%, 127%, and 113% resulting from culturing of HFDP cells in the culturing environment using culturing preparations containing the composition at concentrations of 12.5 nM, 62.5 nM, and 125 nM, respectively. The results are compared to an expression of 108% resulting from culturing using MNX at 10 μM and an expression of 100% resulting from culturing using the vehicle only, which is set as a reference and a negative control.

The data of FIG. 11B suggest that the expression of SHBG may have peaked when the composition is at a concentration of 12.5 nM or below, suggesting a concentration of below 125 nM, below 62.5 nM, below 12.5 nM, etc, would be sufficiently promising.

### Transforming Growth-Interacting Factor (TGIF):

Transforming Growth-Interacting Factor (TGIF) is found to associate with and divert Axin1 and Axin2 from β-catenin destruction complex, allowing β-catenin accrual. It is also found that TGIF represses transcription from an androgen-dependent promoter induced by dihydrotestosterone (DHT) which is a kind of androgens and potentially reduces DHT induced detrimental effect via androgen driven transcription on hair follicle. It is also found that TGIF suppresses both TGF-β- and retinoid-driven gene transcription via binding with smad2 and RARβ respectively.

Example culturing of HFDP cells using selected concentrations of the composition in the culturing environment showed substantial gain in gene expression of TGIF, as evident from FIG. 11C.

Referring to FIG. 11C, the data show expressions of 140%, 146%, and 123% resulting from culturing of HFDP cells in the culturing environment using culturing preparations containing the composition at concentrations of 12.5 nM, 62.5 nM, and 125 nM, respectively. The results are compared to an expression of 124% resulting from culturing using MNX at 10 μM and an expression of 100% resulting from culturing using the vehicle only, which is set as a reference and a negative control.

The data of FIG. 11C suggest that the expression of TGIF may have peaked at a concentration between 12.5 nM and 125 nM, and may have peaked in the vicinity of 62.5 nM, for example, ±5 nM, ±10 nM, ±15 nM, ±20 nM, ±25 nM, ±30 nM of 62.5 nM.

### Variants of LCH34

LCH67 is a variant of the composition (LCH34) which has been shown to be promising and has the below structure.

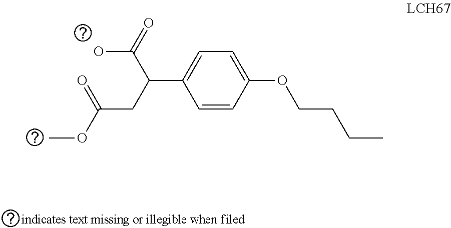 [SMILES: CCCCOc1ccc(C(CC(=O)OC)C(=O)O)cc1]

LCH67, which is 2-(4-Butoxyphenyl) butanedioic acid, has a molecular weight of 266.29 and a SMILES expression of:

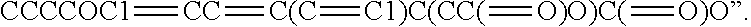

Referring to FIG. 12, LCH67 at concentration of 80 μM and 400 μM enhances gene expression of hair growth markers including ALP and Versican and hair promoting growth factors including PDGF-AA, KGF and FGF2. LCH67 at concentration of 800 μM enhances gene expression of KGF.

LCH64 is a variant of the composition (LCH34) which has been shown to be promising and has the below structure.

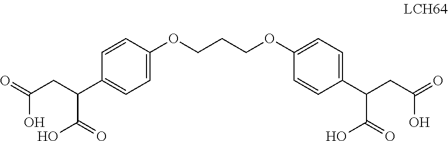 [SMILES: O=C(O)CC(C(=O)O)c1ccc(OCCCOc2ccc(C(CC(=O)O)C(=O)O)cc2)cc1]

LCH64, which is Butanedioic acid, 2,2′-[1,3-propanediylbis(oxy-4,1-phenylene)]bis-, has a SMILES expression of:

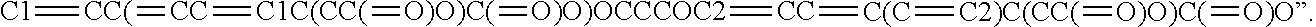

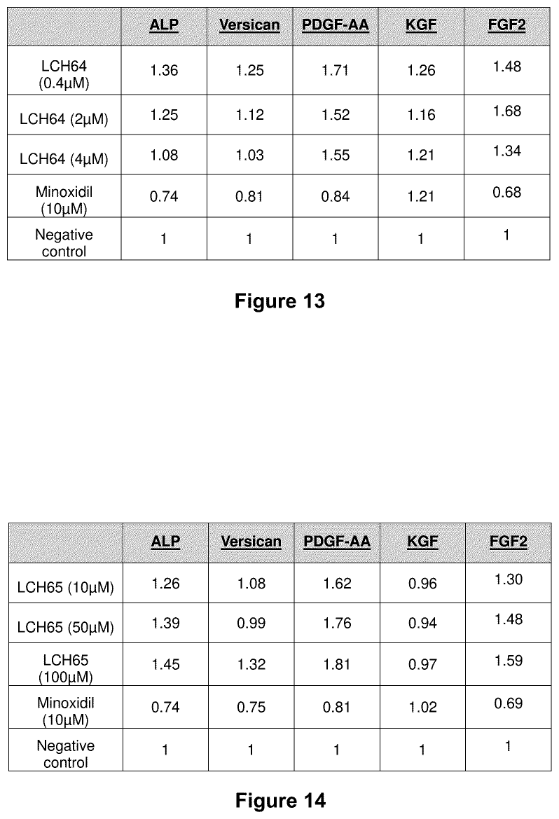

Referring to FIG. 13, LCH64 at concentration of 0.4 μM, 2 μM and 4 μM enhances gene expression of hair growth markers including ALP and Versican and hair promoting growth factors including PDGF-AA, KGF and FGF2.

LCH65 is a variant of the composition (LCH34) which has been shown to be promising and has the below structure.

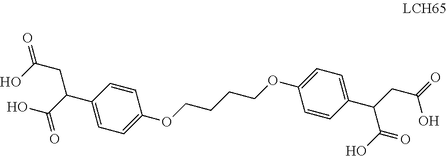 [SMILES: O=C(O)CC(C(=O)O)c1ccc(OCCCCOc2ccc(C(CC(=O)O)C(=O)O)cc2)cc1]

LCH65, which is 2-[4-[4-[4-(1,2-dicarboxyethyl)phenoxy]butoxy]phenyl]butanedioic acid, has a SMILES expression of:

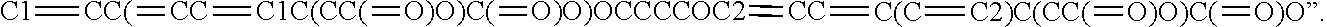

Referring to FIG. 14, LCH65 at concentration of 10 μM and 100 μM enhances gene expression of hair growth markers including ALP and Versican and hair promoting growth factors including PDGF-AA and FGF2. LCH65 at concentration of 50 μM enhance gene expression of ALP, PDGF-AA and FGF2.

While the present disclosure has been made with reference to compositions, agents and preparations that would promote hair growth in human beings, the compositions, agents and preparations for promotion are not limited for promoting hair growth, and may be used for other purposes, for example, as a medication, as a food supplement, or for general health purposes without loss of generality.

### Synthesis of LCH34

An example method for synthesis of LCH34 comprises formation of a first intermediary compound LCH34a and then a second intermediary compound LCH34b.

### 1) Synthesis of LCH34a

The first intermediary compound LCH34a (1,2-Bis(4-cyanomethylphenoxy)ethane, cas number: 34074-31-2) is a bis-cyanomethyl ether derivative, which may be synthesized with reference to the below reactions by, for example using Williamson Ether Synthesis (specifically, a double Williamson ether synthesis or bis-alkylation).

### Preparation of LCH34a:

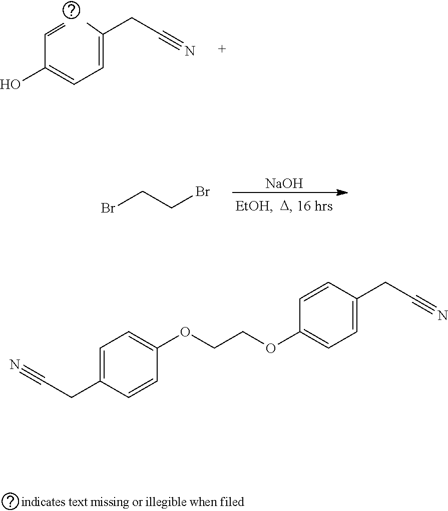

In a 500 mL round-bottom flask, 20 g of 4-hydroxybenzyl cyanide (0.15 mol) was dissolved in 200 mL of 60% ethanol in water. 6 g of solid sodium hydroxide pearl (0.15 mol) was added to this solution, followed immediately by 14.1 g 1,2-dibromoethane (0.0751 mol) in one go. The reaction mixture was then heated to reflux with continuous stirring, for example, in an oil bath for 16 hours under air. Upon completion, the reaction mixture was cooled in an ice bath, inducing product precipitation within minutes. The precipitate was isolated via suction filtration using a Buchner funnel and washed with room-temperature water (25 mL×3) and 95% ethanol (25 mL×3). The resulting solid was dried under vacuum, yielding LCH34a as a brown solid. The final product was obtained with a yield of 52.8% (11.59 g) based on the 4-hydroxybenzyl cyanide. ¹H NMR (300MH, d-DMSO): δ 7.24 (4H, d), 7.00 (4H, d), 4.29 (4H, s), 3.94 (4H, s), as shown in FIG. 19.

Therefore, LCH34a was prepared by a double Williamson ether synthesis between 4-hydroxybenzyl cyanide and 1,2-dibromoethane in aqueous ethanol under basic conditions.

### 2) Synthesis of LCH34b:

The second intermediary compound LCH34b may be synthesized from LCH34a with reference to the below reactions, for example, by alkylation of the cyanomethyl ether with potassium chloroacetate:

From LCH34a to LCH34b:

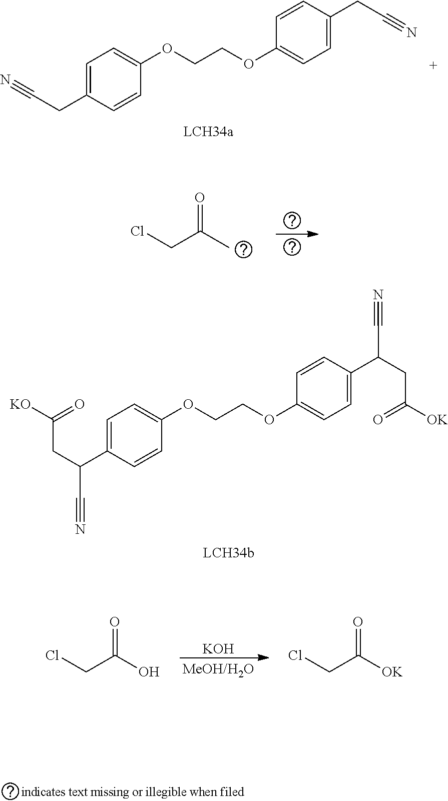

Potassium chloroacetate was freshly prepared by dissolving 6.061 g chloroacetic acid (0.0641 mol) and 3.598 g potassium hydroxide pellet (0.0641 mol) in a mixture of 20 mL methanol and 5 mL water in a 500 mL Schlenk flask under air. The mixture was sonicated for 5 minutes at ambient temperature to ensure complete dissolution, followed by rotary evaporation at 50° C. under reduced pressure to afford a dry white solid of potassium chloroacetate. The dry potassium chloroacetate was then dissolved into 200 mL DMF as received without any further purification process at room temperature. To this solution, 7.5 g LCH34a (0.0257 mol) and an additional 5.758 g potassium hydroxide fine powder (0.1026 mol) were added under a stream of Argon. The reaction mixture was then stirred at room temperature under an Argon atmosphere for 16 hours. The target product was precipitated after 16 hours and isolated via suction filtration by Buchner funnel. The white precipitate was washed with room temperature ethanol (50 mL×3).

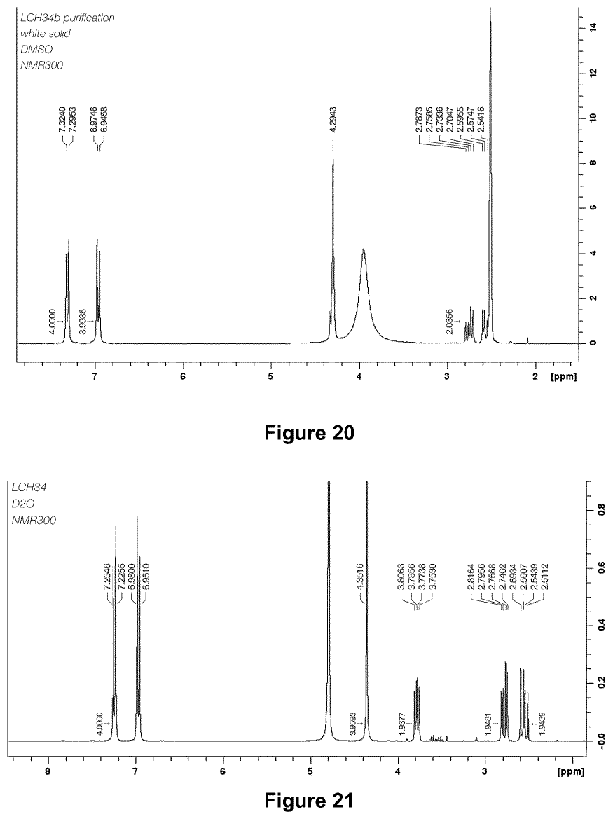

For further purification, the crude LCH34b was first dissolved in 25 mL water, and 10 mL 1 M hydrochloric acid was added dropwise. The white precipitate formed was isolated by suction filtration with Buchner funnel. The final product, LCH34b was then washed by additional ethanol (50 mL×3). Yield: 5.2 g, 42.05%. ¹H NMR (300 MHz, d-DMSO): δ 7.31 (4H, d), 6.96 (4H, d), 4.29 (s, 4H), 2.54-2.78 (4H, dq), as shown in FIG. 20.

3) Synthesis of LCH34 from LCH34b:

Finally, the carboxylate salt LCH34b was subjected to forcing hydrolysis with aqueous sodium hydroxide under reflux to afford the final polyacid product LCH34, which was isolated by precipitation.

From LCH34b to LCH34.

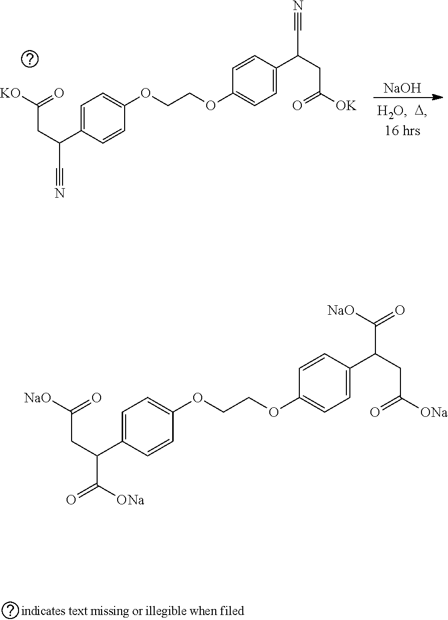

The entirety of the previously synthesized LCH34b (5.2 g) was dissolved in 100 mL of water, followed by the addition of 5.13 g of NaOH pearl (0.128 mol). The resulting mixture was refluxed with stirring in oil bath for 16 hours under air. Without cooling to room temperature, the reaction mixture underwent distillation under reduced pressure to remove 80 mL water. While stirring, the concentrated solution was dropwisely added into 500 mL of room temperature ethanol in a 1 L conical flask. A white solid was precipitated immediately, which was isolated via suction filtration using Buchner funnel. The filtered white solid was washed with room temperature ethanol (50 mL×3), followed by room temperature diethyl ether (50 mL×3). The obtained product was then dried under vacuum to yield the final compound, LCH34. Yield: 5.762 g, 99.3%. ¹H NMR (300 MHz, D₂O): δ 7.24 (4H, d), 6.97 (4H, d), 4.35 (4H, s), 3.75-3.80 (2H, dd), 2.51-2.81 (4H, dq), as shown in FIG. 21.

1. A composition for promoting human and animal wellbeing, including enhancing or promoting hair growth, wherein the composition is a compound having a backbone structure of: 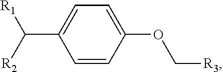
wherein at least one of R1, R2, and R3 is a hydrogen bond donor or a hydrogen bond acceptor.

2. The composition of claim 1, wherein R1, R2 and R3 are selected such that when fitted in the backbone, the compound is to promote growth or proliferation of human hair follicle dermal papilla cells or such that growth or proliferation of human hair follicle dermal papilla cells is promoted, and wherein R1, R2 and R3 are selected such that when fitted in the backbone, the compound is to prolong anagen phase or such that anagen phase is prolonged.

3. The composition of claim 1, wherein R1, R2 and R3 are selected such that when fitted in the backbone, the composition promotes growth or proliferation of and/or or promotion of ALP, Versican, and/or beta-catenin, and wherein R1, R2 and R3 are selected such that when fitted in the backbone, the compound is to induce or enhance expression of sex-hormone-binding globulin, TGIF, TCF7L1, KGF, PDGF and/or bFGF.

4. The composition of claim 1, wherein R1, R2 and R3 are selected such that when fitted in the backbone, the compound is to suppress TGF-β and/or GSK3beta, and wherein R1, R2 and R3 are selected such that when fitted in the backbone, the compound is to activate Wnt pathway, including activating Wnt pathway via GSK3beta/beta-catenin/TCF7L1 in hair follicle dermal papilla cells.

5. The composition of claim 1, wherein R1, R2 and R3 are selected such that when fitted in the backbone, the compound is to not to induce cytotoxicity, including not to induce release of LDH and/or melanin clumping.

6. The compositions of claim 1, wherein R1, R2, and R3 of the backbone structure are, 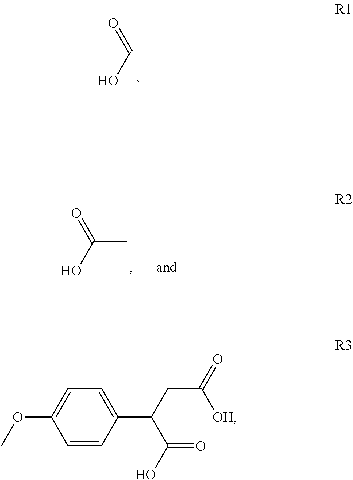
respectively.

7. The composition of claim 1 wherein the composition has a structure of:

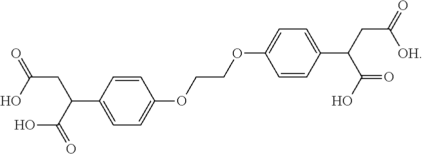 [SMILES: O=C(O)CC(C(=O)O)c1ccc(OCCOc2ccc(C(CC(=O)O)C(=O)O)cc2)cc1]

8. The composition of claim 1, wherein the composition is in a hydrophobic or oil-soluble form, such as an acid form, and/or wherein the composition is in an acid form of: 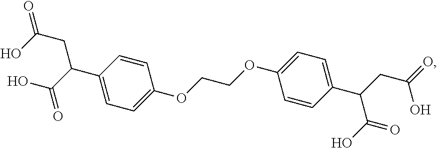 [SMILES: O=C(O)CC(C(=O)O)c1ccc(OCCOc2ccc(C(CC(=O)O)C(=O)O)cc2)cc1]
wherein the acid form in SMILES expression is:

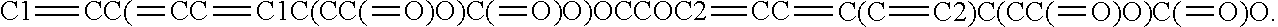

9. The composition of claim 1, wherein the composition is in a hydrophilic or water-soluble form, such as a salt form, and/or wherein the composition is in a sodium salt form of: 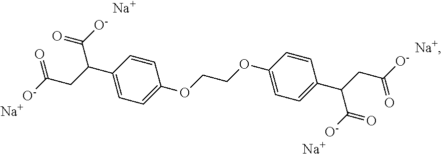
wherein the sodium salt form in SMILES expression is:

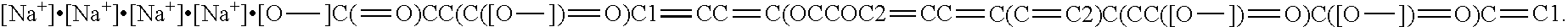

10. The composition of claim 1, wherein the composition is selected from:
a potassium salt form of: 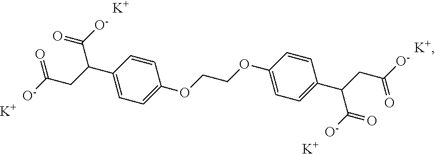
wherein the potassium salt form in SMILES expression being: 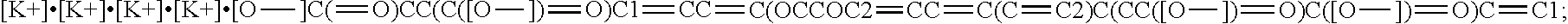
or
an ammonium salt form of: 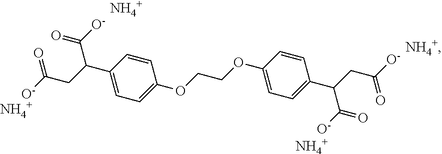
wherein the ammonium salt form in SMILES expression is:

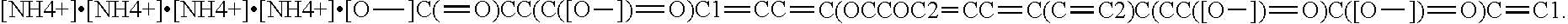

11. The composition of claim 1, wherein the composition is selected from:
2-(4-Butoxyphenyl) butanedioic acid having a structure of: 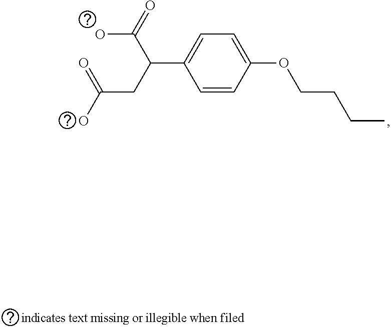 [SMILES: CCCCOc1ccc(C(CC(=O)O)C(=O)O)cc1]
or
a SMILES expression of: CCCCOC1=CC=C(C═C1)C(CC(═O)O)C(═O)O, or Butanedioic acid, 2,2′-[1,3-propanediylbis(oxy-4,1-phenylene)]bis- having a structure of: 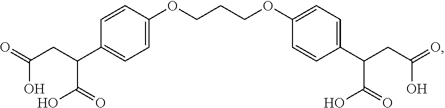 [SMILES: O=C(O)CC(C(=O)O)c1ccc(OCCCOc2ccc(C(CC(=O)O)C(=O)O)cc2)cc1]
with a SMILES expression of:

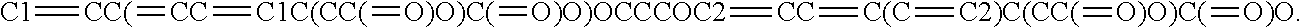

12. The composition of claim 1, wherein the composition is selected from one of 2-[4-[4-[4-(1,2-dicarboxyethyl)phenoxy]butoxy]phenyl]butanedioic acid having a structure of: 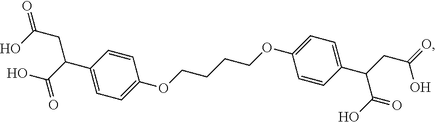 [SMILES: O=C(O)CC(C(=O)O)c1ccc(OCCCCOc2ccc(C(CC(=O)O)C(=O)O)cc2)cc1]
and a SMILES expression of: C1=CC(═CC═C1C(CC(═O)O)C(═O)O)OCCCCOC2=CC═C(C═C2)C(CC(═O)O)C(═O)O, or 2-(4-{2-[4-(1,2-dicarbamoylethyl)phenoxy]ethoxy}phenyl)butanediamide having a structure of: 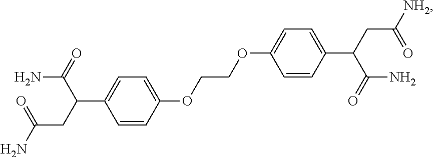 [SMILES: NC(=O)CC(C(N)=O)c1ccc(OCCOc2ccc(C(CC(N)=O)C(N)=O)cc2)cc1]
and a SMILES expression of:

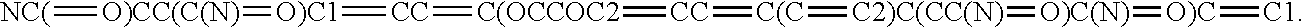

13. The composition of claim 1 wherein the composition is selected from:
22-(4-{2-[4-(1,2-dicarbamimidoylethyl)phenoxy]ethoxy}phenyl)butanebis(imidamide) having a structure of: 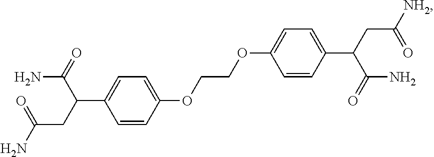 [SMILES: NC(=O)CC(C(N)=O)c1ccc(OCCOc2ccc(C(CC(N)=O)C(N)=O)cc2)cc1]
and a SMILES expression of: 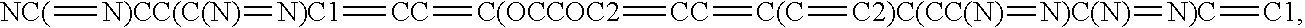
or
2-(4-Butoxy-phenyl)-N-methyl-succinamic acid having a structure of: 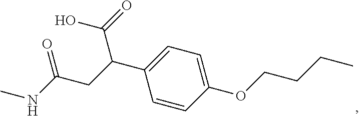 [SMILES: CCCCOc1ccc(C(CC(=O)NC)C(=O)O)cc1]
and a SMILES expression of:

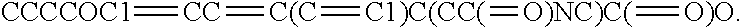

14. The composition of claim 1 wherein the composition is selected from:
3-(4-Butoxyphenyl)hex-4-ynoic acid having a structure of: 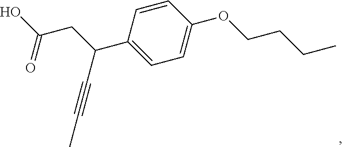 [SMILES: CC#CC(CC(=O)O)c1ccc(OCCCC)cc1]
and a SMILES expression of: 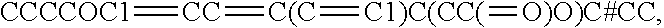
or
2-[4-(Spiro[5.5]undecan-4-ylmethoxy)phenyl]butanedioic acid having a structure of: 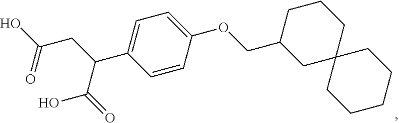 [SMILES: O=C(O)CC(C(=O)O)c1ccc(OCC2CCCC3(CCCCC3)C2)cc1]
and a SMILES expression of:

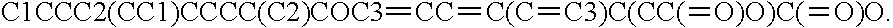

15. The composition of claim 1 wherein the composition is 4,4,4-Trifluoro-3-(4-propoxyphenyl) butanoic acid having a structure of: 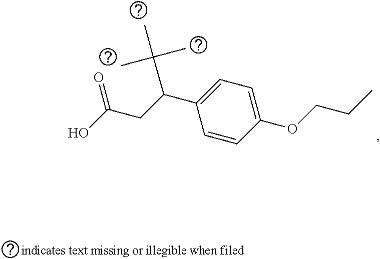 [SMILES: CCCOc1ccc(C(CC(=O)O)C(C)(C)C)cc1]
and a SMILES expression of:

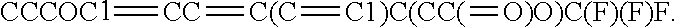

16. A preparation comprising a composition for enhancing or promoting hair growth, wherein the composition is a compound having a backbone structure of: 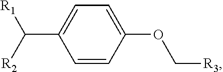
wherein at least one of R1, R2, and R3 is a hydrogen bond donor or a hydrogen bond acceptor,
wherein R1, R2 and R3 are selected such that when fitted in the backbone, the compound is to promote growth or proliferation of human hair follicle dermal papilla cells or such that growth or proliferation of human hair follicle dermal papilla cells is promoted, and
wherein R1, R2 and R3 are selected such that when fitted in the backbone, the compound is to prolong anagen phase or such that anagen phase is prolonged.

17. The preparation of claim 16, wherein the composition has a concentration of 10 μM or higher, including 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, 200 μM, or higher, or a range or ranges selected from a combination of any of the aforesaid values.

18. The preparation of claim 16, wherein the composition has a concentration of 100 μM or higher, including 200 μM, 300 μM, 400 μM, 500 μM, 600 μM, 700 μM, 800 μM, 900 μM, 1 nM, 1.1 nM, 1.2 nM, or higher, or a range or ranges selected from a combination of any of the aforesaid values.

19. The preparation of claim 16, wherein the composition has a concentration of 10 mM or lower, including 9 mM, 8 mM, 7 mM, 6 mM, 5 mM, 4 mM, 3 mM, 2 mM, 1 nm, or lower, or a range or ranges selected from a combination of any of the aforesaid values.

20. The preparation of claim 16, wherein the preparation is aqueous or oil based.
